(19) United States
(12) Patent Application Publication
(10) Pub. No.: US 2007/0258921 A1
(43) Pub. Date: Nov. 8, 2007

(54) PROCESS FOR DEPIGMENTING THE SKIN

(75) Inventor: Maria Dalko, Gif S/Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/741,792

(22) Filed: Apr. 30, 2007

Related U.S. Application Data
(60) Provisional application No. 60/797,389, filed on May 4, 2006.

(30) Foreign Application Priority Data
Apr. 28, 2006 (FR) 06 51520

(51) Int. Cl. A61K 8/60

(52) U.S. Cl. 424/62

(57) ABSTRACT

Process for depigmenting and/or whitening human skin by the application of at least one compound of formula (I):

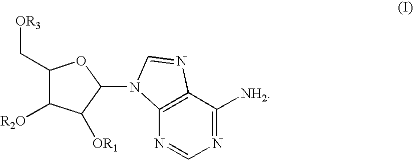

### REFERENCE TO PRIOR APPLICATIONS

This application claims priority to U.S. provisional application 60/797,389 filed May 4, 2006, and to French patent application 0651520 filed Apr. 28, 2006, both incorporated herein by reference.

### FIELD OF THE INVENTION

The present invention relates to a process for depigmenting and/or whitening the skin using an adenosine compound.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The colour of the human skin depends on various factors, in particular on the seasons of the year, race and sex; it is mainly determined by the nature and the concentration of melanin produced by the melanocytes. Melanocytes are specialized cells which synthesize melanin via specific organelles, the melanosomes. In addition, at different periods in their lives, some people witness the appearance on the skin and more especially on the hands of darker and/or more highly coloured blemishes which give the skin a heterogeneous appearance. These blemishes are also due to a high concentration of melanin in the keratinocytes situated at the surface of the skin.

The use of inoffensive topical depigmenting substances which are highly effective is very particularly sought after with a view to treating regional hyperpigmentations by melanocytic hyperactivity, such as idiopathic melasmas, arising during pregnancy (“mask of pregnancy” or chloasma) or oestrone/progestogen contraception, localized hyperpigmentation by benign melanocytic hyperactivity and proliferation, such as senile pigment blemishes known as actinic lentigines, accidental hyperpigmentations, possibly due to photosensitization or to post-lesional healing, as well as certain leucodermas, such as vitiligo. For the latter conditions (healing which can result in a scar giving the skin a whiter appearance), for want of being able to repigment the damaged skin, the end result is to depigment the remaining normal skin regions to give the whole skin a homogeneous white colouring.

The mechanism of formation of the pigmentation of the skin, that is to say of the formation of melanin, is particularly complex and involves, schematically, the following main stages:

Tyrosine→Dopa→Dopaquinone→Dopachrome→Melanin

Tyrosinase (monophenol dihydroxyl phenylalanine: oxygen oxidoreductase EC 1.14.18.1) is the essential enzyme involved in this sequence of reactions. In particular, it catalyses the conversion reaction of tyrosine to give Dopa (dihydroxyphenylalanine), by virtue of its hydroxylase activity, and the conversion reaction of Dopa to give dopaquinone, by virtue of its oxidase activity. This tyrosinase only acts when it is in the maturation state under the effect of certain biological factors.

A substance is recognized as depigmenting if it acts directly on the vitality of the epidermal melanocytes where melanogenesis takes place and/or if it interferes with one of the stages in the biosynthesis of melanin, either by inhibiting one of the enzymes involved in melanogenesis or by being inserted as structural analogue of one of the chemical compounds in the sequence for the synthesis of melanin, which sequence can then be blocked and thus ensure depigmentation.

The substances most commonly used as depigmenting agents are more particularly hydroquinone and its derivatives, in particular its ethers, such as hydroquinone monomethyl ether and monoethyl ether. These compounds, although they exhibit a degree of effectiveness, are not unfortunately devoid of side effects due to their toxicity, which can render their use problematic, indeed even dangerous. This toxicity originates from them interfering in fundamental mechanisms of melanogenesis with the death of the cells, which then risk disrupting their biological environment and which consequently force the skin to discharge them, producing toxins.

Thus, hydroquinone is a particularly irritating compound which is cytotoxic for the melanocyte, the total or partial replacement of which has been envisaged by numerous authors.

A search has thus been undertaken for substances which do not interfere with the mechanism of melanogenesis but which act upstream on tyrosinase by preventing its activation and are for this reason much less toxic. Use is commonly made, as inhibitor of the activation of tyrosinase, of kojic acid, which complexes the copper present in the active site of this enzyme. Unfortunately, this compound is unstable in solution, which somewhat complicates the manufacture of the composition.

The need remains for a novel whitening agent for human skin with an action as effective as those known but not having their disadvantages, that is to say which is non-irritating, non-toxic and/or non-allergizing for the skin while being stable in a composition or else alternatively which has a reinforced action, so as to be able to be used in a smaller amount, which greatly reduces the side effects observed.

### SUMMARY OF THE INVENTION

In this respect, the inventor has discovered, surprisingly and unexpectedly, that certain adenosine compounds exhibit a good depigmenting and/or whitening activity, even at low concentration, without showing cytotoxicity.

### DETAILED DESCRIPTION OF THE PREFERRED EMBODIMENTS

More specifically, one subject-matter of the invention is a process for depigmenting and/or whitening skin, preferably human skin, comprising the application, to the skin, of a at least one compound of formula (I):

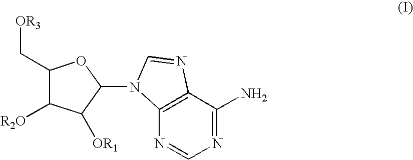

in which:
R1 and R2, which are identical, denote a saturated linear C1-C6 or unsaturated linear C2-C6 or saturated or unsaturated branched C3-C6 hydrocarbon radical or else form, together with the oxygen atoms to which they are attached, an isopropylidene radical;
R3 denotes:

(i) a saturated linear C1-C10 or unsaturated linear C2-C10 or saturated or unsaturated branched C3-C10 hydrocarbon radical optionally substituted by at least one group chosen from —OR′, —NR′R″, —COOR′, —CONR′R″, —CF3, —F, —OCF3, —CN or —NO2;

or (ii) a —COR4 group with R4 denoting a saturated linear C1-C9 or unsaturated linear C2-C9 or saturated or unsaturated branched C3-C9 hydrocarbon radical optionally substituted by at least one group chosen from —OR′, —NR′R″, —COOR′, —CONR′R″, —CF3, —F, —OCF3, —CN or —NO2;

or (iii) an ester group resulting from biotin;

R′ and R″ denote a hydrogen atom or a saturated linear C1-C6 or unsaturated linear C2-C6 or saturated or unsaturated branched C3-C6 hydrocarbon radical optionally substituted by at least one group chosen from —OZ, —NZZ′ or —COOZ, Z and Z′ denoting, independently of one another, a hydrogen atom or a saturated linear C1-C6 or unsaturated linear C2-C6 or saturated or unsaturated branched C3-C6 hydrocarbon radical;

and its salts, optical isomers and solvates.

Preferably, the at least one compound of formula (I) is present in a composition, preferably a cosmetic composition comprising a physiologically acceptable medium.

The compounds of formula (I) according to the invention make it possible to effectively depigment and/or lighten or whiten human skin. They are intended in particular to be applied to the skin of individuals exhibiting brownish pigmentation blemishes or blemishes due to ageing or to the skin of individuals wishing to combat the onset of a brownish colour resulting from melanogenesis, for example following exposure to ultraviolet radiation.

The process is suitable in particular for removing brownish pigmentation blemishes and/or blemishes due to ageing and/or for lightening brown skin.

A further subject-matter of the invention is the use of at least one compound of formula (I) as described above as whitening and/or depigmenting agent for the skin, in particular for removing pigment blemishes or blemishes due to ageing, and/or as antibrowning agents.

Another subject-matter of the invention is the use of at least one compound of formula (I) as described above in the manufacture of a cosmetic or dermatological composition intended to depigment and/or whiten the skin.

Some of the compounds of formula (I) are known from the prior art and are described in the following documents:
WO-A-2004/037159 describes 2′,3′-isopropylidene-5′-acetyladenosine (compound 265, page 203) in a pharmaceutical composition for the treatment of obesity. This document does not describe applying the composition topically to the skin in order to depigment the latter;
Poppe, L et al.; “Synthesis and characterization of (5′-deoxyadenosin-5′-yl)cobalamin (=‘adenosylcobalamin’) analogs mimicking the transition-state geometry of coenzyme-B12-dependent rearrangements” ; Helvetica Chimica Acta (1993), 76(6), 2367-83;
Jones, A. S. et al.; “Synthetic analogs of polynucleotides. VII. Syntheses of 5′-O-acryloyinucleosides and copolymers of these with other acryloyl compounds”; Journal of the Chemical Society [Section] C: Organic (1971), (19), 3183-7;
Mornet, D. et al.; “The reaction of myosin with a bromoalkyl analog of adenosine triphosphate”; FEBS Letters (1977), 84(2), 362-6;
Huber Gerhard; “Esters of adenosine with organic and inorganic acids”; Chem. Ber., 89, 2853-62 (1956)—ref CA52:2027g
Takemoto, K. et al.; “Nucleic acid analogs: their specific interaction and applicability”; Polymeric Materials Science and Engineering (1988), 58, 250-3;
Purkayastha, Bhupesh C.; Bhattacharyya, S. N.; “Use of Ca oxalate monohydrate in the investigation of rare earth and thorium activities”; J. Indian. Chem. Soc., 34, 427-33 (1957) —ref CA52:2627h;
Peterli, Stefan et al.; “Nitrostyrene derivatives of adenosine 5′-glutarates as selective inhibitors of the epidermal growth factor receptor protein tyrosine kinase”; Helvetica Chimica Acta (1992), 75(3), 696-706.
   Those not known in the art make up a part of the invention herein.

The term “alkyl”, in the context of the present invention, means a saturated or unsaturated hydrocarbon chain. Mention may in particular be made, among the alkyl groups suitable for the implementation of the invention, of the methyl, ethyl, isopropyl, n-propyl, n-butyl, t-butyl, isobutyl, sec-butyl, pentyl, n-hexyl, cyclopropyl, cyclopentyl, cyclohexyl or allyl groups.

Mention may be made, as examples of salts of the compounds of formula (I), of the salts obtained by addition of compound of formula (I) with an inorganic acid, chosen in particular from hydrochloric acid, sulphuric acid and phosphoric acid, or with an organic acid, chosen in particular from acetic acid, propionic acid, succinic acid, fumaric acid, lactic acid, glycolic acid, citric acid and tartaric acid. Suitable solvate solvents include water, ethanol, isopropanol, etc. While it may be more technically correct to call the solvates with water “hydrates,” for the purposes of this application they are included wtihin solvates.

Preferably, the salts of the compounds (I) are chosen from the salts obtained from hydrochloric acid or acetic acid or citric acid.

In the formula (I), the hydrocarbon (or alkyl) groups can preferably be chosen in particular, as the case may be, from the groups: methyl, ethyl, n-propyl, isopropyl, n-butyl, isobutyl, tert-butyl, pentyl, hexyl, heptyl, octyl, nonyl or decyl.

Preferred radicals R1 and R2 described above are those which form, together with the oxygen atoms to which they are attached, an isopropylidene radical.

Preference is given, for the compounds of formula (I), to those having the following meanings:
R1 and R2 form, together with the oxygen atoms to which they are attached, an isopropylidene radical;
R3 denotes:

(i) a saturated linear C1-C10 or unsaturated linear C2-C10 or saturated or unsaturated branched C3-C10 hydrocarbon radical;

or (ii) a —COR4 group with R4 denoting a saturated or unsaturated linear C1-C9 or branched C3-C9 hydrocarbon radical;

or (iii) an ester group resulting from biotin.

Use is preferably made of the compounds of formula (I) for which:
R1 and R2 form, together with the oxygen atoms to which they are attached, an isopropylidene radical;
R3 denotes:

(i) a linear C1-C10 hydrocarbon radical;

or (ii) a —COR4 group with R4 denoting a saturated linear C1-C9 hydrocarbon radical;

or (iii) an ester group resulting from biotin.

Use is more preferably made of compounds of formula (I) for which:
R1 and R2 form, together with the oxygen atoms to which they are attached, an isopropylidene radical;
R3 denotes:
a —COR4 group with R4 denoting a linear C1-C9 hydrocarbon radical;
or an ester group resulting from biotin.

The compounds (I) can be prepared according to one of the four synthetic processes described below depending on the meaning of the radicals R1, R2 and R3.

First Process

The compounds of formula (I) for which R1=R2 and R3 denote a hydrocarbon radical as defined above can be prepared in particular according to the following reaction scheme I:

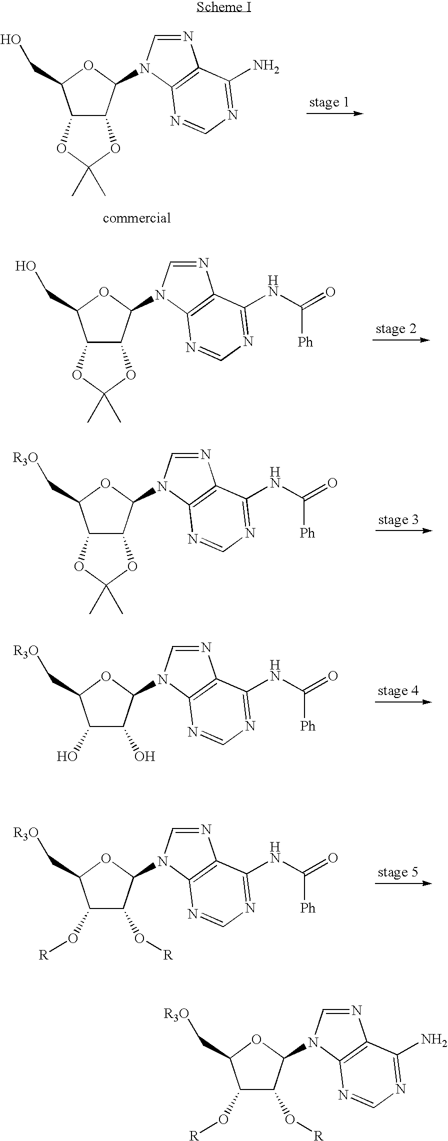

Such a preparation method is described in particular in Helvetica Chimica Acta, 1993 (vol. 76), page 2367.

According to stage 1, isopropylideneadenosine is reacted with chlorotrimethylsilane, in particular in pyridine, and then 1.2 molar equivalents of benzoyl chloride are added. After formation of the corresponding N-benzoyl derivative, sodium fluoride in a water/methanol mixture in an acidic medium is added.

According to stage 2, sodium hydride in dimethylformamide is added to the compound obtained, and a tosylate of formula R3OTs (Ts denotes the tosyl group) or a halogen compound of formula R3X (with X denoting Cl, Br or I) is added.

According to stage 3, a 10% aqueous hydrochloric acid solution and methanol are added and the mixture is brought to reflux for 10 minutes.

According to stage 4, sodium hydride in dimethylformamide and then 2 molar equivalents of tosylate compound of formula R3OTs (Ts denotes the tosyl group) or of halogen compound of formula R3X (with X denoting Cl, Br or I) are added.

According to stage 5, a catalytic amount of sodium methoxide in methanol is added.

Second Process

The compounds of formula (I) for which R1 and R2 together form an isopropylidene radical and R3 denotes a hydrocarbon radical as defined above can be prepared in particular according to the following reaction scheme III:

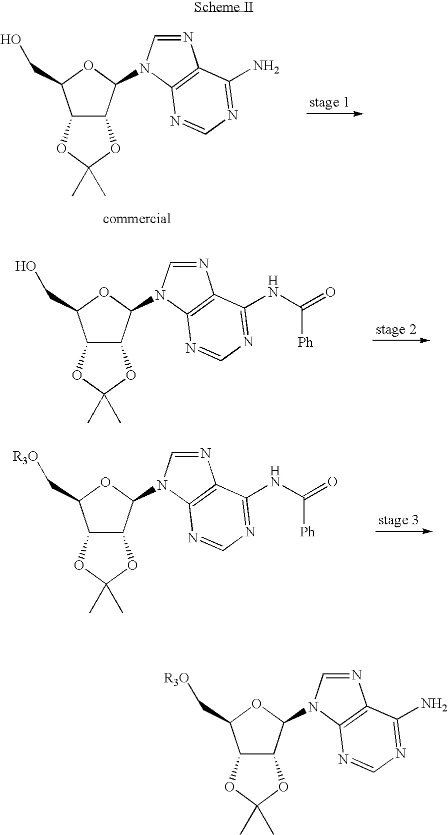

According to stage 1, isopropylideneadenosine is reacted with chlorotrimethylsilane, in particular in pyridine, and then 1.2 molar equivalents of benzoyl chloride are added. After formation of the corresponding N-benzoyl derivative, sodium fluoride in a water/methanol mixture in an acidic medium is added.

According to stage 2, sodium hydride in dimethylformamide is added to the compound obtained and a tosylate of formula R3OTs (Ts denotes the tosyl group) or a halogen compound of formula R3X (with X denoting Cl, Br or I) is added.

According to stage 3, a catalytic amount of sodium methoxide in methanol is added.

Third Process

The compounds of formula (I) for which R1=R2 and denote a hydrocarbon radical and R3 denotes a —COR4 radical or an ester group resulting from biotin as defined above can be prepared in particular according to the following reaction scheme II:

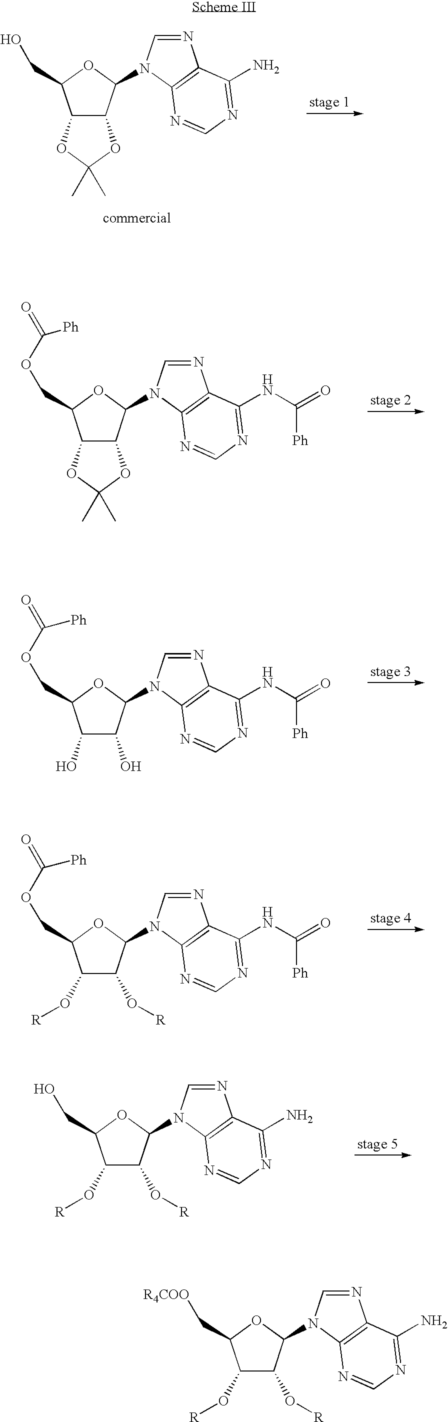

According to stage 1, isopropylideneadenosine is reacted with 2.1 molar equivalents of benzoyl chloride, in particular in pyridine.

Then, according to stage 2, a 10% aqueous hydrochloric acid solution and methanol are added and the mixture is brought to reflux for 10 minutes.

According to stage 3, sodium hydride in dimethylformamide and then 2 molar equivalents of halogen compound of formula R3X (with X denoting Cl, Br or I) are added.

According to stage 4, a catalytic amount of sodium methoxide in methanol is added.

According to stage 5, an organic acid of formula R4COOH is added in the presence of carbonyidiimidazole in dimethylformamide at a temperature of approximately 40° C.

Fourth Process

The compounds of formula (I) for which R1 and R2 together form an isopropylidene radical and R3 denotes a —COR4 radical or an ester group resulting from biotin as defined above can be prepared in particular according to the following reaction scheme IV:

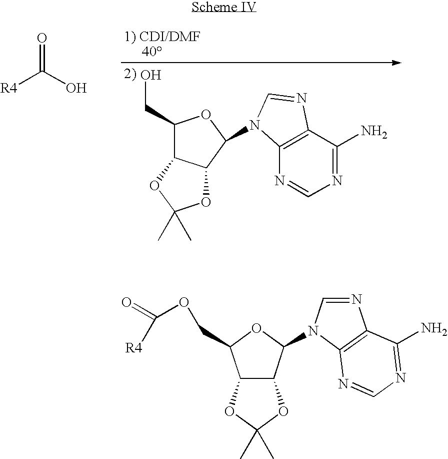

The carboxylic acid R4COOH is reacted in the presence of carbodiimide (CDI) in dimethylformamide at a temperature of approximately 40° C. and isopropylideneadenosine is added.

Particular mention may be made, as compounds of formula (I), of the following compounds:

Compound A: 2′,3′-isopropylidene-5′-butanoyladenosine

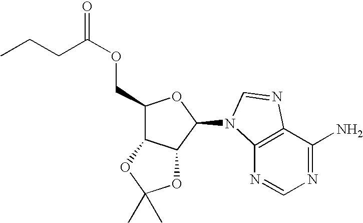

Compound B: 2′,3′-isopropylidene-5′-octanoyladenosine

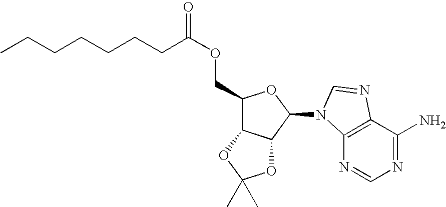

Compound C: 2′,3′-isopropylidene-5′-biotinoyladenosine

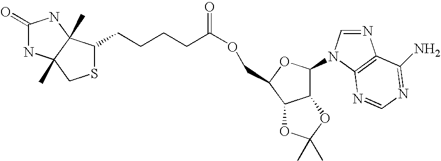

Compound D: 2′,3′-isopropylidene-5′-ethyladenosine

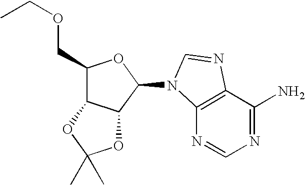

Compound E: 2′,3′-isopropylidene-5′-octyladenosine

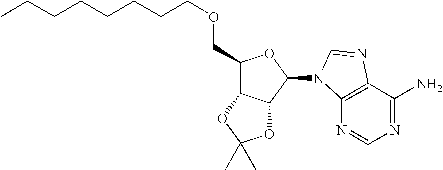

Compound F: 2′,3′-dimethyl-5′-ethyladenosine

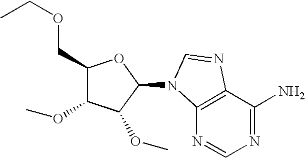

Compound G: 2′,3′-dimethyl-5′-butanoyladenosine

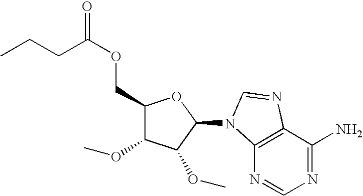

Compound H: 2′,3′-isopropylidene-5′-acetyladenosine (CAS No 15888-38-7)

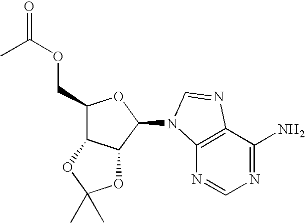

The composition for the use according to the invention is of course preferably suitable for topical application to the skin. The physiologically acceptable medium will preferably be a cosmetically or dermatologically acceptable medium, that is to say without an unpleasant odour, colour or appearance and which does not cause smarting, tightness or redness unacceptable to the user.

The term “physiologically acceptable medium” is understood to mean a medium compatible with human keratinous substances, such as the skin, mucous membranes, nails, scalp and/or hair.

The composition for the use according to the invention can be intended for a cosmetic or pharmaceutical, particularly dermatological, application. Preferably, the composition according to the invention is intended for a cosmetic application.

The amount of compounds of formula (I) which can be used in the context of the invention depends on the effect desired. By way of example, this amount can range, for example, from 0.001% to 10% by weight, preferably from 0.01% to 5% by weight, in particular from 0. 1% to 2% by weight, with respect to the total weight of the composition applied.

The composition can then comprise all the constituents conventionally employed in the application envisaged.

Mention may in particular be made of water, solvents, oils of mineral, animal and/or vegetable origin, waxes, pigments, fillers, surfactants, cosmetic or dermatological active principles, UV screening agents, polymers, gelling agents or preservatives.

Of course, a person skilled in the art will take care to choose this or these optional additional compounds and/or their amounts so that the advantageous properties of the compounds according to the invention are not, or not substantially, detrimentally affected by the envisaged addition.

The composition for the use according to the invention can be provided in any formulation form, including those normally used in the cosmetic and dermatological fields; it can in particular be in the form of an aqueous or aqueous/alcoholic solution which is optionally gelled, of a dispersion of the optionally two-phase lotion type, of an oil-in-water or water-in-oil or multiple emulsion, of an aqueous gel, of a dispersion of oil in an aqueous phase using spherules, it being possible for these spherules to be polymer nanoparticles, such as nanospheres and nanocapsules, or better still lipid vesicles of ionic and/or nonionic type.

When the composition for the use according to the invention is an emulsion, the proportion of the fatty phase can range for example from 5 to 80% by weight and preferably from 5 to 50% by weight, with respect to the total weight of the composition. The oils, the emulsifiers and the optional coemulsifiers used in the composition in the emulsion form are chosen from those conventionally used in the field under consideration. The emulsifier and the coemulsifier are typically present in the composition in a proportion ranging from 0.3 to 30% by weight and preferably from 0.5 to 20% by weight, with respect to the total weight of the composition.

This composition can be more or less fluid and have the appearance of a white or coloured cream, of an ointment, of a milk, of a lotion, of a serum, of a paste or of a foam. It can optionally be applied to the skin in the aerosol form. It can also be provided in the solid form, for example in the stick form.

This composition can for example constitute a cleansing, protective, treatment or care cream for the face, for the hands, for the feet, for the major anatomical folds or for the body (for example, day creams, night creams, make-up-removing creams, foundation creams or sunscreens), a liquid foundation, a make-up-removing milk, a protective or care body milk, a sun milk or a lotion, gel or foam for caring for the skin, such as a cleansing lotion.

In an advantageous aspect of the invention, the compositions used can additionally comprise at least one desquamating agent and/or at least one soothing agent and/or at least one organic photoprotective agent and/or at least one inorganic photoprotective agent.

The term “desquamating agent” is understood to mean any compound capable of acting:
either directly on desquamation by promoting exfoliation, such as β-hydroxy acids, in particular salicylic acid and its derivatives (including 5-(n-octanoyl)salicylic acid); α-hydroxy acids, such as glycolic acid, citric acid, lactic acid, tartaric acid, malic acid or mandelic acid; urea; gentisic acid; oligofucoses; cinnamic acid; Saphora japonica extract; resveratrol;
or on the enzymes involved in desquamation or decomposition of the corneodesmosomes, such as glycosidases, stratum corneum chymotryptic enzyme (SCCE) or indeed even other proteases (trypsin, chymotrypsin-like). Mention may be made of agents which chelate inorganic salts: EDTA; N-acyl-N,N′,N′-ethylene-diaminetriacetic acid; aminosulphonic compounds and in particular 4-(2-hydroxyethyl)piperazine-1-propane sulphonic acid (HEPES); 2-oxothiazolidine-4-carboxylic acid (procysteine) derivatives; derivatives of α-amino acids of glycine type (as disclosed in EP-0 852 949, and also the sodium methylglycinediacetate sold by BASF under the trade name Trilon M); honey; or sugar derivatives, such as O-octanoyl-6-D-maltose and N-acetylglucosamine.

Particular mention may be made, as soothing agents which can be used in the composition according to the invention, of: pentacyclic triterpenes and plant extracts (for example, Glycyrrhiza glabra) comprising them, such as p-glycyrrhetinic acid and its salts and/or its derivatives (glycyrrhetinic acid monoglucuronide, stearyl glycyrrhetinate or 3-stearoyloxyglycyrrhetic acid), ursolic acid and its salts, oleanolic acid and its salts, betulinic acid and its salts, an extract of Paeonia suffruticosa and/or lactiflora, salts of salicylic acid and in particular zinc salicylate, phycosaccharides from Codif, an extract of Laminaria saccharina, canola oil, bisabolol, camomile extracts, allantoin, Sepivital EPC (phosphoric diester of vitamin E and C) from Seppic, omega-3 unsaturated oils, such as musk rose, blackcurrant seed, echium or fish oils, plankton extracts, capryloylglycine, Seppicalm VG (sodium palmitoylproline and Nymphaea alba) from Seppic, an extract of Pygeum, an extract of Boswellia serrata, an extract of Centipeda cunninghami, an extract of Helianthus annuus, an extract of Linum usitatissimum, tocotrienols, extracts of Cola nitida, piperonal, an extract of clove, an extract of Epilobium angustifolium, aloe vera, an extract of Bacopa monnieri, phytosterols, cortisone, hydrocortisone, indomethacin and betamethasone.

The organic photoprotective agents are preferably chosen in particular from anthranilates; cinnamic derivatives; dibenzoylmethane derivatives; salicylic derivatives; camphor derivatives; triazine derivatives, such as those disclosed in Patent Applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bis-benzoazolyl derivatives, such as disclosed in Patents EP 669 323 and U.S. Pat. No. 2,463,264; p-aminobenzoic acid (PABA) derivatives; methylenebis(hydroxyphenyl benzotriazole) derivatives, such as disclosed in Applications U.S. Pat. No. 5,237,071, U.S. Pat. No. 5,166,355, GB 2 303 549, DE 197 26 184 and EP 893 119; and screening polymers and screening silicones, such as those disclosed in particular in Application WO 93/04665; dimers derived from α-alkylstyrene, such as those disclosed in Patent Application DE 198 55 649.

The inorganic photoprotective agents are preferably chosen from pigments or alternatively nanopigments (mean size of the primary particles: generally between 5 nm and 100 nm, preferably between 10 nm and 50 nm) formed of coated or noncoated metal oxides, such as, for example, titanium oxide (amorphous or crystalline in the rutile and/or anatase form), iron oxide, zinc oxide, zirconium oxide or cerium oxide nanopigments, which are all UV photoprotective agents well known per se. Conventional coating agents are furthermore alumina and/or aluminium stearate. Such nanopigments, formed of coated or noncoated metal oxides, are disclosed in particular in Patent Applications EP 518 772 and EP 518 773.

The photoprotective agents are generally present in the composition according to the invention in proportions ranging from 0.1 to 20% by weight, with respect to the total weight of the composition, and preferably ranging from 0.2 to 15% by weight, with respect to the total weight of the composition.

The examples which follow illustrate the invention without limiting the scope thereof. The compounds are, as the case may be, cited under the chemical names or under the CTFA (International Cosmetic Ingredient Dictionary and Handbook) names.

### EXAMPLE 1

### Synthesis of 2′,3′-isopropylidene-5′-biotinoyladenosine (Compound C)

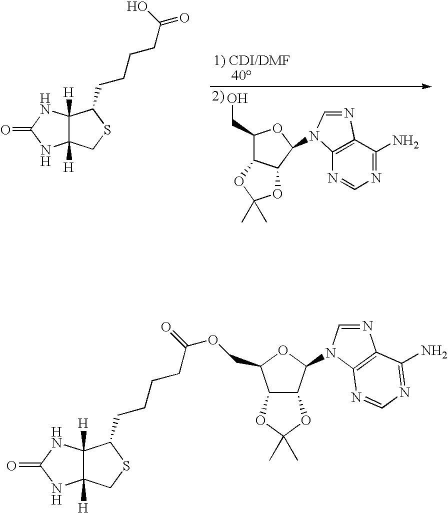

150 ml of dimethylformamide, 5 g of biotin and then 4 g of carbonyidiimidazole (recorded as CDI) were introduced into a three-necked flask under nitrogen. The mixture was heated at 40° C. for 1 hour, then 6.3 g of commercial isopropylideneadenosine were added and then 5 g of sodium amide were added. The solution was heated at 40° C. for 24 hours.

The dimethylformamide (recorded as DMF) was distilled off without exceeding 40° C. and 200 ml of dichloromethane were added to the residue.

The organic phase was washed 3 times with 150 ml of water, dried with sodium sulphate and evaporated under vacuum to dryness.

The residue was purified on a silica column, eluent dichloromethane then dichloromethane 95/methanol 5, to result in a solid product which was taken up in dichloromethane and precipitated with diethyl ether.

The precipitate obtained was filtered off, washed with ether and dried under vacuum.

Yield=50%

Analyses:

NMR DMSO 1H 13C 2D: Spectra in accordance

Elemental analysis in accordance: C 51.3; H 5.89; N 18.2; O 19.03; S 5.92

### EXAMPLE 2

### Synthesis of 2′,3′-isopropylidene-5′-butanoyladenosine (Compound A)

This compound is prepared according to a procedure similar to that of Example 1 using butyric acid in place of biotin.

### EXAMPLE 3

### Synthesis of 2′,3′-isopropylidene-5′-octanoyladenosine (Compound B)

This compound is prepared according to a procedure similar to that of Example 1 using octanoic acid in place of biotin.

### EXAMPLE 4

### Demonstration of the Activity with Regard to Melanogenesis

A biological test demonstrated the depigmenting activity of the compound of Example 2 (Compound A) and of the known compound H.

The modulating effect with regard to melanogenesis of the compounds tested was measured according to the method described in Patent FR-A-2 734 825 and in the paper by R. Schmidt, P. Krien and M. Régnier, Anal. Biochem., 235(2), 113-18, 1996. This test is carried out on cocultures of keratinocytes and of melanocytes.

For the compound tested, the following were determined:

the cytotoxicity, by estimating the incorporation of leucine,

the inhibitory activity with regard to the synthesis of melanin, by estimating the ratio of the incorporation of thiouracil to the incorporation of leucine, with respect to 100% of the control (the control corresponds to the test carried out without compound to be tested). The IC50 (concentration at which the synthesis of melanin is inhibited by 50%) values were determined.

The results are collated in the following table:

| | | IC50 |
|---|---|---|
| | Cytotoxicity | (μM) |
| Compound Ex. 1 | No | 7.4 |
| Compound H | No | 35 |

The compound of Example 1 and the compound H are thus effective in inhibiting melanogenesis.

The test was also carried out with adenosine, which does not have a significant depigmenting activity (at the concentration of 100 μM, melanin synthesis is inhibited by only 20%).

### EXAMPLE 5

A whitening cream for caring for the face of oil-in-water emulsion type is prepared, comprising (% by weight):

| | |
|---|---|
| compound of Example 2 | 0.005% |
| glycerol stearate | 2% |
| polysorbate 60 (Tween 60 from ICI) | 1% |
| stearic acid | 1.4% |
| triethanolamine | 0.7% |
| carbomer | 0.4% |
| liquid fraction of shea butter | 12% |
| perhydrosqualene | 12% |
| antioxidant | 0.05% |
| fragrance, preservative | q.s. |
| water | q.s. for 100% |

A similar composition is prepared with the compound of Example 1.

### EXAMPLE 6

A depigmenting gel for the skin is prepared, comprising (% by weight):

| | |
|---|---|
| compound H | 2% |
| hydroxypropylcellulose (Klucel H from Hercules) | 1% |
| antioxidant | 0.05% |
| isopropanol | 40% |
| fragrance, preservative | q.s. |
| water | q.s. for 100% |

A similar composition is prepared with the compound of Example 3.

The above written description of the invention provides a manner and process of making and using it such that any person skilled in this art is enabled to make and use the same, this enablement being provided in particular for the subject matter of the appended claims, which make up a part of the original description and including a process for depigmenting and/or whitening human skin comprising the application, to the skin, of a cosmetic composition comprising, in a physiologically acceptable medium, at least one compound of following formula (I):

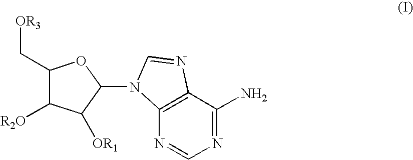

in which:
R1 and R2, which are identical, denote a saturated linear C1-C6 or unsaturated linear C2-C6 or saturated or unsaturated branched C3-C6 hydrocarbon radical or else form, together with the oxygen atoms to which they are attached, an isopropylidene radical;
R3 denotes:

(i) a saturated linear C1-C10 or unsaturated linear C2-C10 or saturated or unsaturated branched C3-C10 hydrocarbon radical optionally substituted by at least one group chosen from —OR′, —NR′R″, —COOR′, —CONR′R″, —CF3, —F, —OCF3, —CN or —NO2;

or (ii) a —COR4 group with R4 denoting a saturated linear C1-C9 or unsaturated linear C2-C9 or saturated or unsaturated branched C3-C9 hydrocarbon radical optionally substituted by at least one group chosen from —OR′, —NR′R′, —COOR′, —CONR′R′, —CF₃, —F, —OCF3, —CN or —NO2;

or (iii) an ester group resulting from biotin;

R′ and R″ denoting a hydrogen atom or a saturated linear C1-C6 or unsaturated linear C2-C6 or saturated or unsaturated branched C3-C6 hydrocarbon radical optionally substituted by at least one group chosen from —OZ, —NZZ′ or —COOZ, Z and Z′ denoting, independently of one another, a hydrogen atom or a saturated linear C1-C6 or unsaturated linear C2-C6 or saturated or unsaturated branched C3-C6 hydrocarbon radical;

and its salts, optical isomers and solvates.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

As noted above, the invention method and novel compounds and compositions are preferably used by subjects desirous of the benefits noted herein, subjects “in need of” these benefits. Such subjects typically have brownish pigmentation blemishes or blemishes due to ageing or wish to combat the onset of a brownish colour resulting from melanogenesis, for example following exposure to ultraviolet radiation or wish to remove brownish pigmentation blemishes and/or blemishes due to ageing and/or who wish to lighten brown skin.

Naturally, one using the invention as disclosed will use an amount of the invention composition effective to provide the noted benefit(s). Such amount is inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for depigmenting and/or whitening human skin comprising applying to skin in need of depigmenting and/or whitening a composition comprising, in a physiologically acceptable medium, at least one compound of following formula (I): 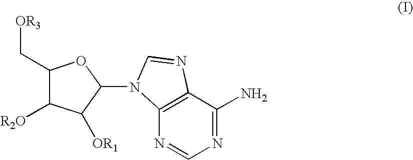
in which:
R1 and R2, which are identical, denote a saturated linear C1-C6 or unsaturated linear C2-C6 or saturated or unsaturated branched C3-C6 hydrocarbon radical or else form, together with the oxygen atoms to which they are attached, an isopropylidene radical;
R3 denotes:
(i) a saturated linear C1-C10 or unsaturated linear C2-C10 or saturated or unsaturated branched C3-C10 hydrocarbon radical optionally substituted by at least one group chosen from —OR′, —NR′R″, —COOR′, —CONR′R″, —CF3, —F, —OCF3, —CN or —NO2;
or (ii) a —COR4 group with R4 denoting a saturated linear C1-C9 or unsaturated linear C2-C9 or saturated or unsaturated branched C3-C9 hydrocarbon radical optionally substituted by at least one group chosen from -OR′, —NR′R″, —COOR′, —CONR′R″, —CF3, —F, —OCF3, —CN or —NO2;
or (iii) an ester group resulting from biotin;
R′ and R″ denoting a hydrogen atom or a saturated linear C1-C6 or unsaturated linear C2-C6 or saturated or unsaturated branched C3-C6 hydrocarbon radical optionally substituted by at least one group chosen from —OZ, —NZZ′ or —COOZ, Z and Z′ denoting, independently of one another, a hydrogen atom or a saturated linear C1-C6 or unsaturated linear C2-C6 or saturated or unsaturated branched C3-C6 hydrocarbon radical;
and salts, optical isomers and solvates thereof.

2. The process according to claim 1, wherein said composition comprises at least one compound of formula (I) where:
R1 and R2 form, together with the oxygen atoms to which they are attached, an isopropylidene radical;
R3 denotes:
(i) a saturated linear C1-C10 or unsaturated linear C2-C10 or saturated or unsaturated branched C3-C10 hydrocarbon radical;
or (ii) a —COR4 group with R4 denoting a saturated or unsaturated linear C1-C9 or branched C3-C9 hydrocarbon radical;
or (iii) an ester group resulting from biotin.

3. The process according to claim 1, wherein said composition comprises at least one compound of formula (I) where:
R1 and R2 form, together with the oxygen atoms to which they are attached, an isopropylidene radical;
R3 denotes:
(i) a linear C1-C10 hydrocarbon radical;
or (ii) a —COR4 group with R4 denoting a saturated linear C1-C9 hydrocarbon radical;
or (iii) an ester group resulting from biotin.

4. The process according to claim 1, wherein said composition comprises at least one compound of formula (I) where:
R1 and R2 form, together with the oxygen atoms to which they are attached, an isopropylidene radical;
R3 denotes:
a —COR4 group with R4 denoting a linear C1-C9 hydrocarbon radical;
or an ester group resulting from biotin.

5. The process according to claim 1, wherein said composition comprises at least one compound selected from the group consisting of:
2′,3′-isopropylidene-5′-butanoyladenosine
2′,3′-isopropylidene-5′-octanoyladenosine
2′,3′-isopropylidene-5′-biotinoyladenosine
2′,3′-isopropylidene-5′-ethyladenosine
2′,3′-isopropylidene-5′-octyladenosine
2′,3′-dimethyl-5′-ethyladenosine
2′,3′-dimethyl-5′-butanoyladenosine, and
2′,3′-isopropylidene-5′-acetyladenosine.

6. The process according to claim 1, wherein the at least one compound of formula (I) is present in an amount of 0.001% to 10% by weight with respect to the total weight of the composition.

7. The process according to claim 5, wherein said composition comprises 2′,3′-isopropylidene-5′-butanoyladenosine.

8. The process according to claim 5, wherein said composition comprises 2′,3′-isopropylidene-5′-octanoyladenosine.

9. The process according to claim 5, wherein said composition comprises 2′,3′-isopropylidene-5′-biotinoyladenosine.

10. The process according to claim 5, wherein said composition comprises 2′,3′-isopropylidene-5′-ethyladenosine.

11. The process according to claim 5, wherein said composition comprises 2′,3′-isopropylidene-5′-octyladenosine.

12. The process according to claim 5, wherein said composition comprises 2′,3′-dimethyl-5′-ethyladenosine.

13. The process according to claim 5, wherein said composition comprises 2′,3′-dimethyl-5′-butanoyladenosine.

14. The process according to claim 5, wherein said composition comprises 2′,3′-isopropylidene-5′-acetyladenosine.

15. The process according to claim 5, wherein the at least one compound of formula (I) is present in an amount of 0.001% to 10% by weight with respect to the total weight of the composition.
